(19) United States
(12) Patent Application Publication
(10) Pub. No.: US 2026/0325756 A1
(43) Pub. Date: Oct. 22, 2026

(54) A SUBASSEMBLY OF A MEDICAMENT DELIVERY DEVICE

(71) Applicant: SHL Medical AG, Zug (CH)

(72) Inventor: Anders Boström, Nacka Strand (SE)

(21) Appl. No.: 19/104,759

(22) PCT Filed: Aug. 8, 2023
(86) PCT No.: PCT/EP2023/071878

§ 371 Date: Feb. 19, 2025

(30) Foreign Application Priority Data
Aug. 22, 2022 (EP) 22191417.9

(51) Int. Cl. A61M 5/20; A61M 5/31; A61M 5/32

(52) U.S. Cl. CPC A61M 5/2033; A61M 5/3146; A61M 5/3202; A61M 2005/2073

(57) ABSTRACT

The present disclosure generally relates to medicament delivery devices such as autoinjectors, and particularly concerns a subassembly for activation and priming of a medicament delivery device.

### CROSS-REFERENCE TO RELATED APPLICATIONS

The present application is a U.S. National Phase Application pursuant to 35 U.S.C. § 371 of International Application No. PCT/EP2023/071878 filed Aug. 8, 2023, which claims priority to EP Patent Application No. 22191417.9 filed Aug. 22, 2022. The entire disclosure contents of these applications are herewith incorporated by reference into the present application.

### TECHNICAL FIELD

The present disclosure generally relates to medicament delivery devices such as autoinjectors, and particularly concerns a subassembly of a medicament delivery device.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology. Not least amongst these are the difficulties faced by patients that require frequent injections and by patients that need to inject particularly viscous drugs. In considering these problems, the applicant has appreciated that various developments could be made to help improve the medicament delivery devices on the market today, for example concerning activation and priming of the medicament delivery devices prior to injection, which are set out in more detail below.

### SUMMARY

An object of the present disclosure is to provide a subassembly for a medicament delivery device which solves, or at least mitigates problems of the prior art.

According to a first aspect of the present disclosure, there is provided a subassembly of a medicament delivery device, the subassembly comprising: a housing extending in a longitudinal direction from a proximal end to a distal end relative a longitudinal axis, a rear knob comprising a distal portion protruding out from the distal end of the housing and a proximal portion arranged in the housing, the proximal portion comprising a flexible arm having a protrusion extending radially inwards, the protrusion comprising a distally facing surface; a plunger rod movable in the housing and being configured to expel medicament from a medicament container, the plunger rod comprising a proximally facing surface configured to abut against the distally facing surface of the rear knob; a plunger rod spring arranged inside the plunger rod; an activation body arranged to surround at least part of the plunger rod and the proximal portion of the rear knob, the activation body comprising a surface facing radially inwards that is configured to be in contact with the flexible arm of the rear knob to prevent the flexible arm from moving radially outwards when the rear knob is in an initial position, wherein the rear knob is axially movable relative to the activation body in a distal direction away from the initial position, whereby an abutting contact between the distally facing surface of the rear knob and the proximally facing surface of the plunger rod causes the plunger rod to be moved in the distal direction when the rear knob is moved, thereby moving the flexible arm away from the surface to a position where the flexible arm is allowed to flex radially outwards and release the plunger rod.

Embodiments of the present disclosure advantageously provides for a release mechanism for the plunger before priming by a distal motion of the rear knob. The distal motion of the rear knob is preferably achieved by a pulling action performed by a user prior to an injection event. The engagement between the plunger rod and the rear knob causes the plunger rod to follow a distal motion of the rear knob, until the flexible arm of the rear knob is moved sufficiently to be released and allowed to flex radially outwards. As a result, the engagement between the plunger rod and the rear knob is released so that the plunger rod can be pushed proximally by the plunger spring towards an activation position in which the plunger rod causes a pressure build up in a syringe prior to priming.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction 301 relative to an axis 102, typically a central axis extending in the direction of the longest extension of the device and/or component. Similarly, “radial” or “radially” refer to a direction 302 extending radially relative to the axis, and “rotation”, “rotational” and “rotationally” refer to rotation relative to the axis.

According to one embodiment, the distally facing surface of the rear knob may be inclined with respect to the longitudinal axis. The inclined surface facilitates the radially outwards flexing motion of the flexible arm by interaction with the proximally facing surface of the plunger rod so that the plunger rod can be released.

According to one embodiment, the proximally facing surface of the plunger rod may be inclined with respect to the longitudinal axis. As above, the inclined surface the radially outwards flexing motion of the flexible arm.

According to one embodiment, the rear knob comprises a distally facing surface on a protrusion extending radially outwards, and the activation body comprises a proximally facing surface, wherein the rear knob is blocked from moving further distally from a stop position by engagement between the distally facing surface of the rear knob and the proximally facing surface of the activation body. The stop position advantageously prevents excessive distal motion of the rear knob and for improve user-friendliness of the medicament delivery device.

According to one embodiment, an inner diameter of the activation body at the surface may be smaller than an inner diameter of the activation body at a surface distally adjacent to the surface. This advantageously provides for allowing the flexible arm to move radially outwards once the rear knob has moved sufficiently distally so that a proximal end portion of the flexible arm has moved away from the larger inner diameter and reached the smaller inner diameter of the activation body.

According to one embodiment, the activation body may comprise a protrusion on a flexible arm, the protrusion extending radially inwards towards the plunger rod, and wherein once the plunger rod is released, the plunger rod spring is adapted to push the plunger rod proximally to a primer position where the proximally facing surface of the plunger rod abuts against a distally facing surface of the protrusion of the activation body. The plunger rod is thus firstly released and subsequently held in a priming position before a subsequent injection event.

According to one embodiment, the subassembly may comprise a lock ring arranged radially outside the flexible arm of the activation body to initially prevent the flexible arm from moving radially outwards. The lock ring is preferably a separate part that that is axially movable in relation to the activation body. The lock ring provides a simple yet efficient locking mechanism for the flexible arm of the activation body.

According to one embodiment, the subassembly may comprise a needle cover arranged in the housing and configured to cover a needle at the proximal end of the housing, the needle cover is axially movable in relation to the housing along the longitudinal axis between a distal position and a proximal position, wherein the needle cover is arranged to distally move the lock ring when the needle cover moves towards the distal position. In other words, the motion of the needle cover advantageously causes the locking ring to move away from a locking position so that the flexible arm of the activation body is allowed to flex radially outwards.

According to one embodiment, a distal end of the needle cover may comprise a distally facing surface configured to engage with a proximally facing surface of the locking ring. This engagement enables for the needle cover to move the lock ring in the distal direction.

According to one embodiment, the locking ring may be adapted to distally move a distance away from the flexible arm so that the flexible arm is allowed to move radially outwards so that the plunger rod is allowed to move proximally by the plunger rod spring.

According to one embodiment, the lock ring may comprise an internal spring attached to a distal end of the lock ring. The internal spring causes the locking ring to move proximally when the needle cover moves to its proximal position.

According to one embodiment, the internal spring may be unloaded prior to moving the needle cover towards the distal position.

According to one embodiment, the distally facing surface of the protrusion of the activation body may be inclined with respect to the longitudinal axis.

According to one embodiment, the activation body and the housing may be separate parts. This may be preferable for ease of assembly and/or ease of component manufacture. In this embodiment, the activation body is arranged in the housing.

According to one embodiment, the activation body may be an integral part of the housing. Thus, the activation body may be part of the housing. This may be preferable to minimise the number of components.

According to one embodiment, the distal portion of the rear knob may be accessible to a user for pulling the rear knob in the distal direction.

There is further provided a medicament delivery device comprising the subassembly of any of the herein disclosed embodiments.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an autoinjector according to embodiments of the present disclosure;

FIG. 2 is an exploded view of an autoinjector according to embodiments of the present disclosure;

FIG. 3 is an exploded view of a subassembly according to embodiments of the present disclosure;

FIG. 4 is a perspective view of a rear knob according to embodiments of the present disclosure;

FIG. 5 is a perspective view of a plunger rod according to embodiments of the present disclosure;

FIG. 6 is a perspective view of an activation body according to embodiments of the present disclosure;

FIG. 7 is a cross-sectional view of a perspective view of an activation body according to embodiments of the present disclosure;

FIG. 8 is a perspective view of a lock ring according to embodiments of the present disclosure;

FIG. 9 is a perspective view of a needle cover according to embodiments of the present disclosure;

FIG. 10 is a cross-section of the subassembly in an initial state according to embodiments of the present disclosure;

FIG. 11 is a cross-section of the subassembly when the rear knob has moved distally and the plunger rod has been released according to embodiments of the present disclosure;

FIG. 12 is a cross-section of the proximal part of the medicament delivery device 1 in the activation position;

FIG. 13 is a cross-section of the proximal part of the medicament delivery device 1 in the priming position;

FIG. 14 is a cross-section of the subassembly in a primer position according to embodiments of the present disclosure; and

FIG. 15 is a cross-section of the subassembly in an injection position.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

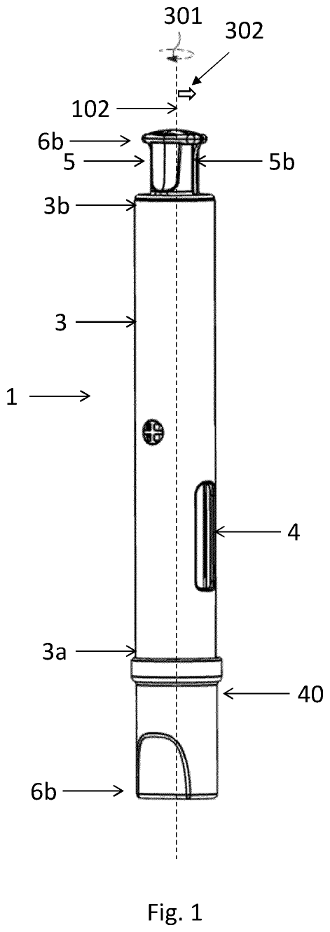

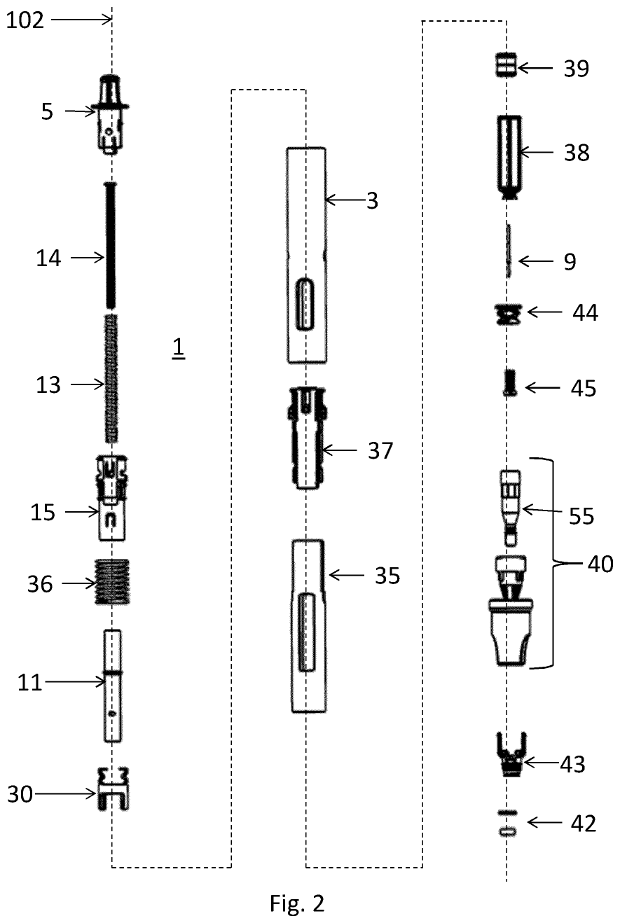

FIG. 1 shows an example of a medicament delivery device 1 such as an autoinjector according to embodiments of the present disclosure and FIG. 2 is an exploded view of the medicament delivery device 1. The medicament delivery device 1 is configured to expel medicament from a medicament container 38 via a medicament delivery member such as a needle 9, to a user at a dose delivery site. The medicament delivery device 1 extends from a proximal end la to a distal end 1b relative to the axis 102.

The medicament delivery device 1 comprises a housing 3 with a window 4. The housing 3 has a proximal end 3a and a distal end 3b. A cap 40 covers the proximal end 3a of the housing 3.

The medicament delivery device 1 further comprises a needle cover 35 arranged in the housing 3 and extends proximally from the proximal end 3a. The needle cover 35 is configured to be moved linearly relative to the housing 3 from an extended position to a retracted position in which the needle cover 35 is received further in the housing 3 and in which a medicament delivery member such as a needle 9 is exposed.

The needle cover 35 is biased in the proximal direction towards the extended position by a spring 36.

A plunger rod 11 is adapted to move in the medicament container to force a medicament out from the medicament container 38 by pushing on a stopper 39 movable within the medicament container 38.

A plunger rod spring 13 guided by a guide rod 14 is arranged inside the plunger rod 11 and is configured to move the plunger rod 11 in the proximal direction to move the stopper 39 in the medicament container 38.

The medicament delivery device comprises several further components that will not be described in detail, such as a cartridge housing 37, sealants 42, a retainer 43, interlock 44, and a needle hub 45.

The medicament delivery device 1 comprises the subassembly 2 which will now be described in more detail with reference to subsequent drawings.

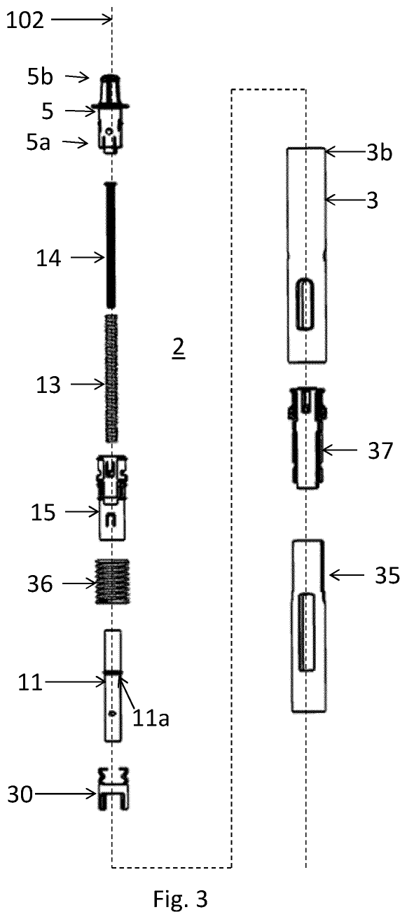

An exploded view of the subassembly is shown in FIG. 3. The subassembly 2 comprises the housing 3 extending in a longitudinal direction 102 from a proximal end 3a to a distal end 3b relative a longitudinal axis 102.

A rear knob 5 comprising a distal portion 5b protruding out from the distal end 3b of the housing 3, also shown in FIG. 1. The rear knob 5 also comprises a proximal portion 5a arranged in the housing 3.

Furthermore, the subassembly 2 comprises a plunger rod 11 which is axially movable in the housing 3 and that is configured to expel medicament from a medicament container 38. The plunger rod 11 comprises a proximally facing surface 11a configured to abut against a distally facing surface 7a of the rear knob 5 as will be described in more detail below. A plunger rod spring 13 arranged inside the plunger rod 11.

An activation body 15 is arranged to surround at least part of the plunger rod 11 and the proximal portion 5a of the rear knob 5.

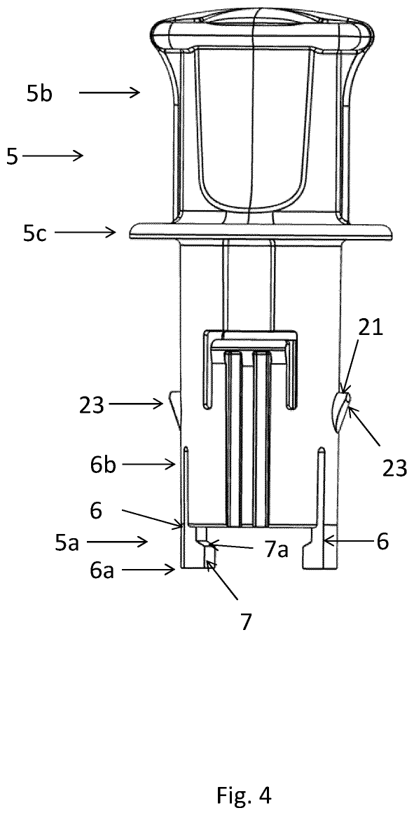

FIG. 4 is a perspective view of the rear knob 5. The proximal portion 5a of the rear knob 5 comprises a flexible arm 6 having a protrusion 7 extending radially inwards. The protrusion 7 comprises a distally facing surface 7a located on a free end 6a of the flexible arm which is also the proximal end of the flexible end. The flexible arm 6 further comprises a fixed end 6b at the distal end of the flexible arm 6. The free end 6a moves in relation to the fixed end 6b when the flexible arm 6 flexes.

The flexible arm 6 is partly formed from a cut-out in the body of the rear knob and protrudes proximally from the body of the rear knob 5.

The distal portion 5b of the rear knob 5 is arranged outside the housing 3 so that a user can access the distal portion 5b, formed as a handle, to pull the rear knob 5 in the distal direction. The flange 5c abuts on the housing 3 and generally defines the proximal-most part of the distal portion 5b.

The distally facing surface 7a is part of the protrusion 7. In order to facilitate forcing the free end 6a of the flexible arm 6 radially outwards by interaction with a proximally facing surface 11a of the plunger rod, the distally facing surface 7a is inclined with respect to the longitudinal axis 102. The inclination is so that the more distal part of the inclined surface 7a is the radially outer-most part of the surface 7a, and the radially inner-most surface portion of the inclined surface 7a is relatively more proximal.

Further, the rear knob 5 comprises a distally facing surface 21 on a protrusion 23 extending radially outwards. The protrusion 23 is more distally location than the flexible arm but is still on the proximal portion 5a of the rear knob.

The rear knob 5 comprises two equal flexible arms 6 located on opposite sides of the rear knob 5. Further, the rear knob 5 comprises two protrusions 23 located on opposite sides of the rear knob 5.

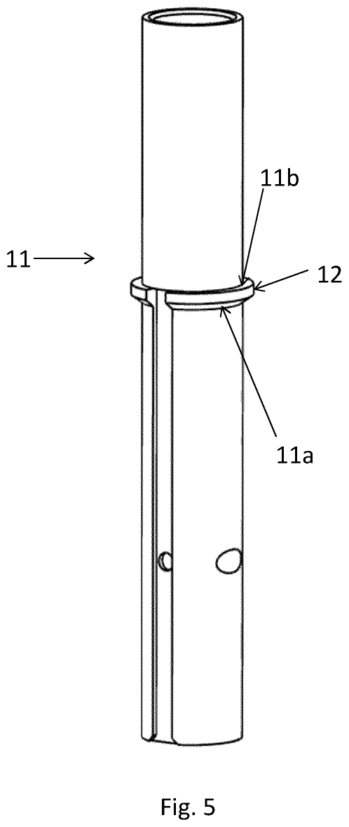

FIG. 5 is a perspective view of the plunger rod 11. The plunger rod 11 is tubular and is configured to receive the plunger rod spring 13 and the guide rod 14 inside its tubular body. The plunger rod 11 comprises a proximally facing surface 11a configured to abut against a distally facing surface 7a of the rear knob 5. The proximally facing surface 11a is on one side of a protrusion 12 shaped like a flange extending around the outer circumference of the plunger rod, but not necessarily circumventing the entire circumference. The proximally facing surface 11a of the plunger rod 11 is inclined with respect to the longitudinal axis 102 in correspondence with the inclined surface 7a of the rear knob to facilitate the motion of the flexible arm 6 radially outwards. The opposite side of the protrusion 12 defines a distally facing surface 11b that is not inclined with respect to the longitudinal axis 102.

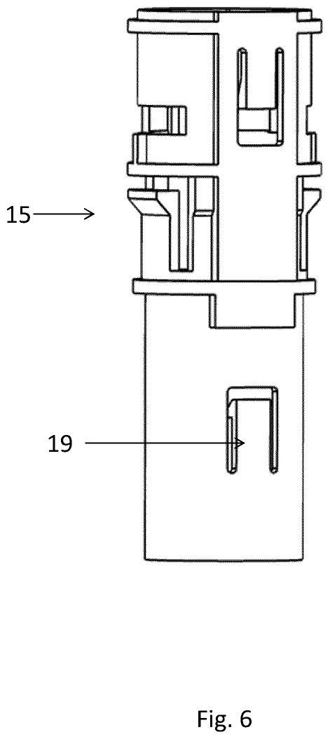

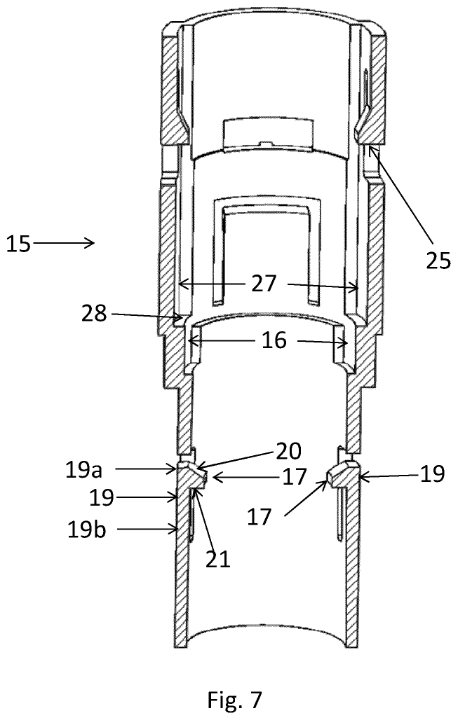

FIG. 6 is a perspective view of the activation body 15 and FIG. 7 is a perspective cross-sectional view of the activation body 15. Here, the activation body 15 is described as a separate part to the housing 11. However, it is also envisaged that the activation body 15 is an integral part of the housing 11.

The activation body 15 comprises a surface 16 facing radially inwards that is configured to be in contact with the flexible arm 6 of the rear knob 5 to prevent the flexible arm 6 from moving radially outwards when the rear knob 5 is in an initial position.

Further, the activation body 15 comprises a proximally facing surface 25. The rear knob 5 is blocked from moving further distally from a stop position by engagement between the distally facing surface 21 of the rear knob 5 and the proximally facing surface 25 of the activation body 15. Thus, in the stop position the distally facing surface 21 comes in physical contact with the proximally facing surface 25 of the activation body 15. The proximally facing surface may preferably be parallel with the distally facing surface 21. The proximally facing surface 25 is here located on a distal end portion of the activation body 15 although this depends on the specific implementation at hand.

The activation body 15 is hollow to receive the plunger rod 11 and the rear knob 5. An inner diameter of the activation body 15 at the surface 16 configured to prevent the radial flexion of the flexible arm 6 of the rear knob 5 is smaller than an inner diameter of the activation body 15 at a surface 27 distally adjacent to the surface 16. An edge 28 may connected the surfaces 26 and 27, where the edge 28 is a distally facing surface located axially between the surfaces 16 and 27.

The activation body 15 comprises a protrusion 17 on a flexible arm 19. The flexible arm 19 comprises a free end 19a and a fixed end 19b where the free end 19a is a distal end of the flexible arm 19 and the fixed end is the proximal end of the flexible arm 19. The free end 19a moves in relation to the fixed end 19b when the flexible arm 19 flexes. The protrusion 17 extending radially inwards towards the plunger rod 11. Furthermore, the protrusion 17 comprises a distally facing surface 20 that is inclined with respect to the axis 102. On the opposite side of the protrusion 17 there is a proximally facing surface 21 that is preferably not inclined with respect to the axis 102. A normal axis of the proximally facing surface 21 may be parallel with the axis 102.

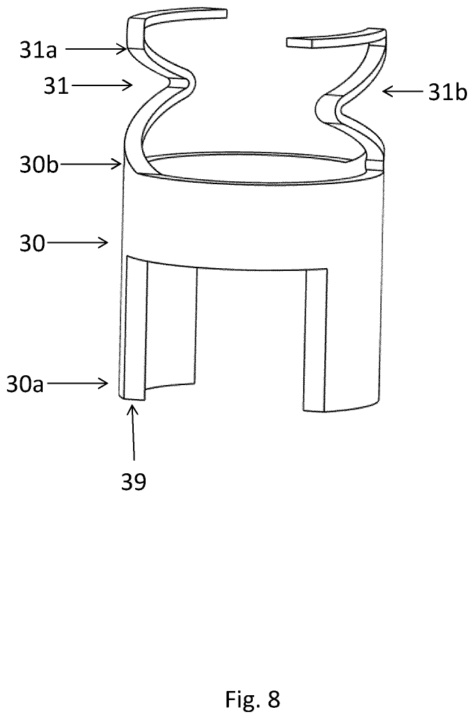

FIG. 8 is a perspective view of a lock ring 30 of the subassembly 2. The lock ring 30 comprises a proximally facing surface 39 of a proximal end 30a of the lock ring 30. The lock ring 30 comprises an internal spring 31 attached to a distal end 30b of the lock ring 30. The internal spring comprises two flexible, preferably plastic, spring elements 31a and 31b that may be integrally formed with the lock ring 30. The spring elements 31a and 31b extend from the distal end 30b of the lock ring and in a distal direction. Alternatively, a spring (i.e. a separate spring, so a separate component) could be used to provide this function instead of the spring elements 31a and 31b. The spring could be metal or plastic, for example. The spring could be arranged between the rear end of the lock ring and the activation body, for example.

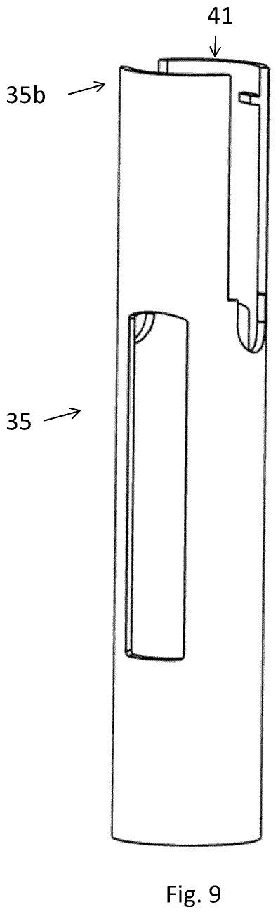

FIG. 9 is a perspective view of a needle cover 35 of the subassembly 2. The needle cover 35 is arranged in the housing 3 and is configured to cover a needle 9 at the proximal end 3a of the housing 3. The needle cover 35 is axially movable in relation to the housing 3 along the longitudinal axis 102 between a distal position and a proximal position. The needle cover 35 is arranged to distally move the locking ring 30 when the needle cover 35 moves towards the distal position.

The needle cover 35 comprises distal end 35b with a distally facing surface 41 configured to engage with the proximally facing surface 39 of the locking ring 30. The needle cover 35 is tubular.

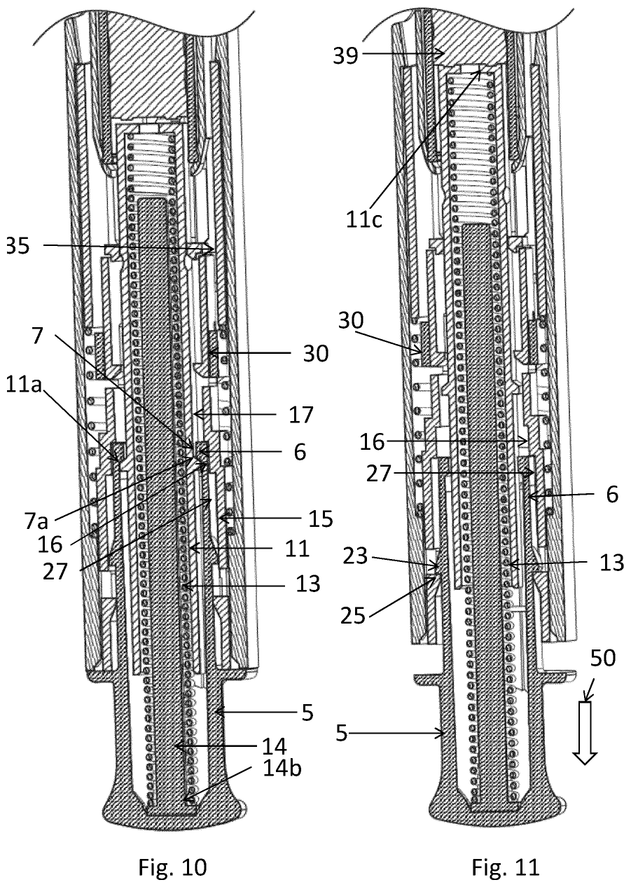

FIG. 10 is a cross-section of the subassembly 2 in an initial state. Here, the flexible arm 6 of the rear knob 5 abuts against the surface 16 of the activation body 15 which prevents the flexible arm 6 to flex radially outwards. Further, the distally facing surface 7a of the rear knob 5 abuts against the proximally facing surface 11a of the plunger rod 11.

When the rear knob 5 is moved axially in relation to the activation body 15 in a distal direction 50 away from the initial position, the abutting contact between the inclined surface 7a of the rear knob 5 and the inclined surface 11a of the plunger rod first causes the plunger rod 11 to be moved linearly in the distal direction 50, as illustrated in FIG. 11. As a result of the distal motion of the rear knob 5, the flexible arm 6 moves away from the surface 16 of the activation body 15 and is instead aligned with an adjacent surface 27 of the activation body 15 where the inner diameter is larger than at surface 16. Here, the flexible arm 6 is allowed to move radially outwards away from axis 102. In other words, the interaction between the inclined surfaces 7a and 11a now causes the flexible arm 6 to flex radially outwards, thereby the contact between the inclined surfaces 7a and 11a is lost and the plunger rod 11 is released and can be pushed by the spring 13 in the proximal direction to an activation position where the plunger rod 11 pushes the stopper 40 proximally inside the medicament container 38 to build up pressure in the medicament container. The plunger rod 11 is maintained in this position by the spring force pushing the plunger rod on the stopper 40 held in place by the pressure in the medicament container.

The spring 13 is biased between the guide rod 14 distal flange 14b that abuts against an inner proximally facing surface the rear knob 5 and an inner distally facing surface 11c of the plunger rod 11.

The distally facing surface 21 of the protrusion 23 of the rear knob 5 contacts the proximally facing surface 25 of the activation body 15 so that the rear rob is stopped from further distal motion.

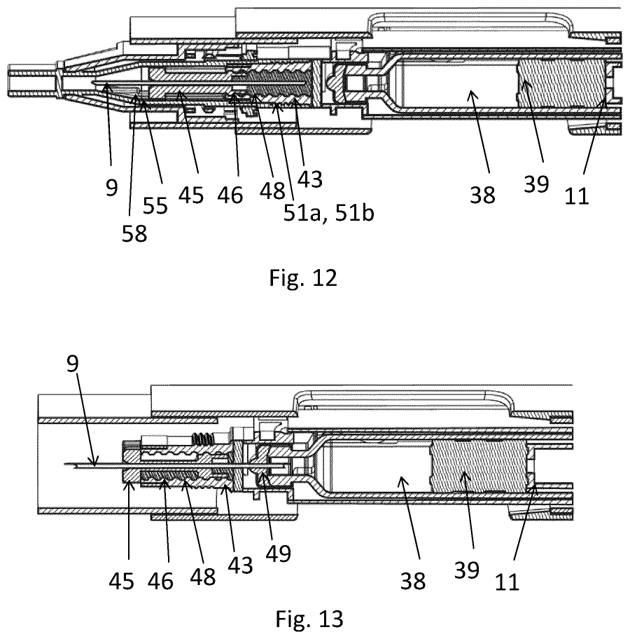

FIG. 12 is a cross-section of the proximal part of the medicament delivery device 1 in the activation position where the plunger rod 11 pushes on the stopper to pressurize the medicament container 38. FIG. 12 shows a needle cap 55, a needle hub 45 holding the needle 9, and a retainer 43. The needle hub 45 has exterior threads 46 that engage with inner threads 48 of the retainer 43. The needle cap 55 and the retainer 43 are engaged at the exterior threads 51a of the retainer and inner threads 51b of the needle cap 55. Further, a mechanical connection between the needle cap 55 and the needle hub 45 prevent axial rotation between them but allow linear motion along axis 102. For example, the needle cap 55 includes a rib 58 and the needle hub has a matching groove. When the needle cap 55 is removed by rotation thereof to unscrew it from the retainer 43 the mechanical connection between the needle cap 55 and the needle hub 45 causes a rotation of the needle hub 45 about the longitudinal axis 102. The rotation of the needle hub 45 and the threaded 46, 48 engagement between the needle hub and the retainer 43 cause the needle hub 45 and the needle 9 to move linearly in the distal direction.

As shown in FIG. 13, the linear motion of the needle hub 45 and the needle 9 lead to the needle 9 reaching into the medicament container 38 through the septum 49 which punctures the medicament container 38. This causes a pressure release in the medicament container 38 so that the plunger rod 11 pushes the stopper 39 further in the proximal direction to cause priming.

Turning to FIG. 12, once the needle cap 55 is removed by a proximal motion as described with reference to FIGS. 12 and 13, the plunger rod 11 is released and the plunger rod spring 13 has pushed the plunger rod 11 proximally to a primer position where the proximally facing surface 11a of the plunger rod 11 abuts against a distally facing surface 20 of the protrusion 17 on the flexible arm 19 of the activation body 15. In the primer position, when the needle 9 has punctuated the medicament container 38, the plunger rod 11 has pushed the stopper 40 further proximally inside the medicament container 38 to eject air from the medicament container 38 in preparation for an injection event. The spring 13 is still biased to push the plunger rod 11 in the proximal direction. However, the lock ring 30 is arranged radially outside the flexible arm 19 of the activation body 15 to initially prevent the flexible arm 19 from moving radially outwards. In this state prior to moving the needle cover towards the distal position, the internal spring 31 (see FIG. 8) of the lock ring 30 is unloaded.

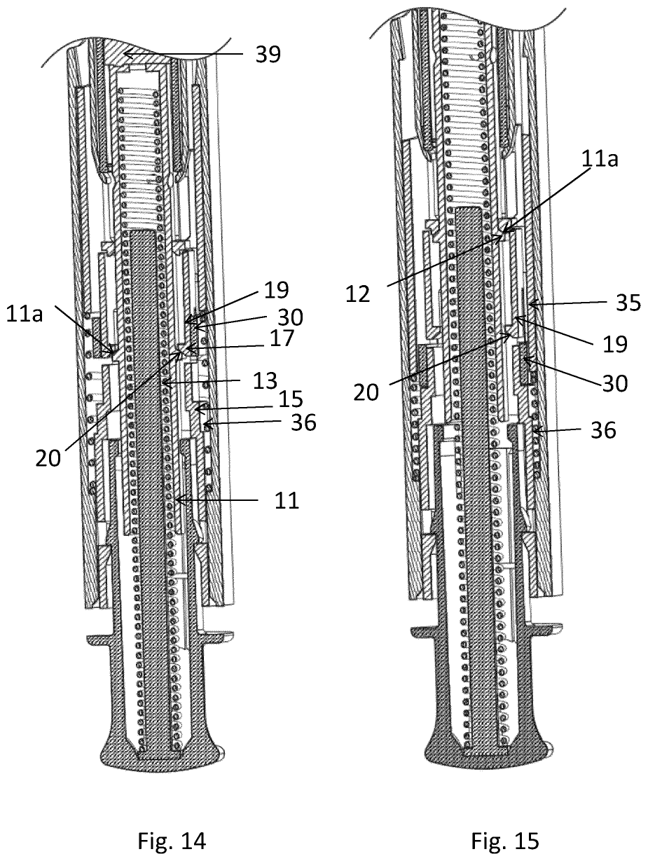

Turning now to FIG. 14, illustrating the subassembly during injection, where the needle cover 35 has moved in the distal direction to compress the needle cover spring 36, the needle cover abuts on the lock ring 30 so that it moves the lock ring 30 distally. The lock ring 30 is moved axially a distance away from the flexible arm 19 so that the flexible arm 19 is allowed to move radially outwards. The interaction between the inclined surfaces 20 and 11a under the influence of the spring force from the plunger rod spring 13 causes the flexible arm 19 to move radially outwards and release the plunger rod 11 so that the plunger rod is allowed to move proximally by the plunger rod spring 13 to push the stopper 40 further in the medicament container 38 to deliver a dose at an injection site.

A medicament delivery device (such as an autoinjector) may generally include various other components. For example, a sensor unit which may recognize injection events, such as the autoinjector inserted into an attachment portion of e.g., a pad, injection started, and injection ends, a memory unit which is configured to store the recorded data during the injection, a connectivity unit configured to transmit the stored data to a smart device or the network directly, a processing unit configured to control the entire system and processes the data before transmitting it, and/or user interface units that are configured to provide feedback to the patient, such as status LEDs, haptic, and/or audio feedback.

When the medicament delivery device is placed into the attachment portion, the sensors inside of the support pad are configured to recognize the event and give feedback to the patient via haptic/visual or audio elements.

When the injection finishes, the sensors are configured to recognize the event and give feedback to the patient again. Further, the collected data is stored in the memory unit and may be transmitted to the smart device/network via the connectivity unit after the injection event finishes.

The sensor can be one of or the combination of the following: a mechanical switch, a Hall-effect sensor, an accelerometer.

The mechanical switch, hall-effect sensor, or accelerometer can be used for detection of the insertion of the auto-injector into an injection port.

The accelerometer can be used for detecting injection events.

Possible wireless communication methods include Bluetooth and Cellular Networks.

Bluetooth connectivity requires a smart device to transmit the stored data to the network and it requires a pairing action between the support pad and the smart device before being able to use the supporting pad. But it's a cheaper alternative and it requires less space on PCB.

The cellular network does not require any pairing process, it can be used as a plug-n-play device, no prior setup is needed, but it's more expensive and it requires more space on PCB.

Depending on the requirements of the product any of those two technologies can be used.

Such processing units may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A subassembly of a medicament delivery device, the subassembly comprising:
a housing extending in a longitudinal direction from a proximal end to a distal end relative a longitudinal axis;
a rear knob comprising a distal portion protruding out from the distal end of the housing and a proximal portion arranged in the housing, the proximal portion comprising a flexible arm having a protrusion extending radially inwards, the protrusion comprising a distally facing surface;
a plunger rod movable in the housing and being configured to expel medicament from a medicament container, the plunger rod comprising a proximally facing surface configured to abut against the distally facing surface of the rear knob;
a plunger rod spring arranged inside the plunger rod; and
an activation body arranged to surround at least part of the plunger rod and the proximal portion of the rear knob, the activation body comprising a surface facing radially inwards that is configured to be in contact with the flexible arm of the rear knob to prevent the flexible arm from moving radially outwards when the rear knob is in an initial position,
wherein the rear knob is axially movable relative to the activation body in a distal direction away from the initial position, whereby an abutting contact between the distally facing surface of the rear knob and the proximally facing surface of the plunger rod causes the plunger rod to be moved in the distal direction when the rear knob is moved, thereby moving the flexible arm away from the surface to a position where the flexible arm is allowed to flex radially outwards and release the plunger rod.

17. The subassembly according to claim 16, wherein the distally facing surface of the rear knob is inclined with respect to the longitudinal axis.

18. The subassembly according to claim 16, wherein the proximally facing surface of the plunger rod is inclined with respect to the longitudinal axis.

19. The subassembly according to claim 16, wherein the rear knob comprises a distally facing surface on a protrusion extending radially outwards, and the activation body comprises a proximally facing surface, wherein the rear knob is blocked from moving further distally from a stop position by engagement between the distally facing surface of the rear knob and the proximally facing surface of the activation body.

20. The subassembly according to claim 16, wherein an inner diameter of the activation body at the surface is smaller than an inner diameter of the activation body at a surface distally adjacent to the surface.

21. The subassembly according to claim 16, wherein the activation body comprises a protrusion on a flexible arm, the protrusion extending radially inwards towards the plunger rod, and wherein once the plunger rod is released, the plunger rod spring is adapted to push the plunger rod proximally to a primer position where the proximally facing surface of the plunger rod abuts against a distally facing surface of the protrusion of the activation body.

22. The subassembly according to claim 21, comprising a lock ring arranged radially outside the flexible arm of the activation body to initially prevent the flexible arm from moving radially outwards.

23. The subassembly according to claim 22, comprising a needle cover arranged in the housing and configured to cover a needle at the proximal end of the housing, the needle cover is axially movable in relation to the housing along the longitudinal axis between a distal position and a proximal position, wherein the needle cover is arranged to distally move the locking ring when the needle cover moves towards the distal position.

24. The subassembly according to claim 23, wherein a distal end needle cover comprises a distally facing surface configured to engage with a proximally facing surface of the locking ring.

25. The subassembly according to claim 23, wherein the locking ring is adapted to distally move a distance away from the flexible arm so that the flexible arm is allowed to move radially outwards so that the plunger rod is allowed to move proximally by the plunger rod spring.

26. The subassembly according to claim 22, wherein the lock ring comprises an internal spring attached to a distal end of the lock ring.

27. The subassembly according to claim 26, wherein the internal spring is unloaded prior to moving the needle cover towards the distal position.

28. The subassembly according to claim 21, wherein the distally facing surface of the protrusion of the activation body is inclined with respect to the longitudinal axis.

29. The subassembly according to claim 16, wherein the activation body and the housing are separate parts, or wherein the activation body is an integral part of the housing.

30. A medicament delivery device comprising the subassembly according to claim 16.
